(19) United States
(12) Patent Application Publication
(10) Pub. No.: US 2026/0347311 A1
(43) Pub. Date: Nov. 5, 2026

(54) DEVICE FOR PRODUCING ISOCYANATE BY LIGHT SOLVENT METHOD AND PRODUCTION METHOD THEREOF

(71) Applicants: Qingdao University of Science and Technology, Qingdao (CN); Qingdao Shenwo Chemical Technology Co., Ltd., Qingdao (CN)

(72) Inventors: Rongshan Bi, Qingdao (CN); Pan Xu, Qingdao (CN); Jijun Ge, Qingdao (CN); Tiezhen Du, Qingdao (CN)

(21) Appl. No.: 19/558,630

(22) Filed: Mar. 6, 2026

(30) Foreign Application Priority Data
Mar. 7, 2025 (CN) 202510273377.2

(51) Int. Cl. C07C 263/20; C07C 263/16

(52) U.S. Cl. CPC C07C 263/20; C07C 263/16

(57) ABSTRACT

A device for producing isocyanate by a light solvent method and a production method thereof. The device includes a pre-fractionation column and a main column. A top and kettle of the pre-fractionation column are respectively in communication with a top and kettle of the main column. The method includes: conveying an isocyanate photochemical reaction material to a pre-fractionation column for preliminary separation; extracting phosgene and a solvent from the top of the pre-fractionation column and then entering the top of the main column; extracting a solvent and isocyanate from the kettle of the pre-fractionation column and then entering the kettle of the main column, separating phosgene from the top of the main column, extracting a liquid-phase solvent from a side line of the main column, and conveying a crude isocyanate product separated from the kettle of the main column to the next process.

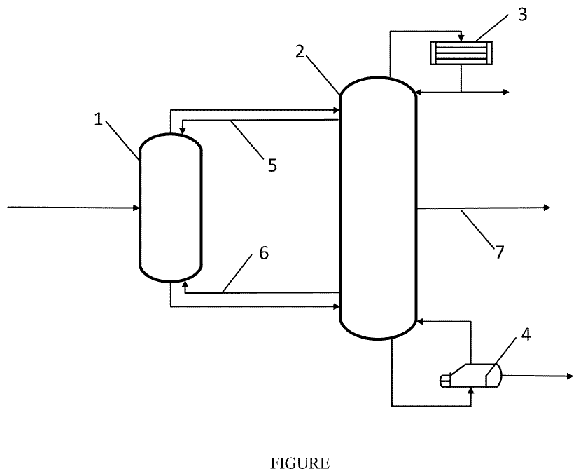

### CROSS-REFERENCE TO RELATED APPLICATIONS

This patent application claims the benefit and priority of Chinese Patent Application No. 202510273377.2 filed with the China National Intellectual Property Administration on Mar. 7, 2025, the disclosure of which is incorporated by reference herein in its entirety as part of the application.

### TECHNICAL FIELD

The present disclosure belongs to the technical field of chemical recycling, and in particular, relates to a device for producing isocyanate by a light solvent method and a production method thereof.

### BACKGROUND

Toluene diisocyanate (TDI) belongs to isocyanate with a molecular formula of C₉H₆N₂O₂. TDI is transparent to light yellow liquid with high reactivity, and is particularly prone to undergo addition reactions with polyols to generate polyurethane. TDI is soluble in benzene, toluene, ethyl acetate, acetone, methyl ethyl ketone, and carbon tetrachloride, but insoluble in organic solvents such as acetonitrile, polyethylene glycol, n-hexane, and petroleum ether. The structure contains two isocyanate groups (-NCO) and a benzene ring, and has high non-polarity and specific polar characteristics. With excellent reactivity and wide application fields, TDI has become a key raw material for producing a high-performance material, an adhesive, and a fiber. TDI is internationally recognized as a fine chemical product featuring the so-called “two larges and one high” characteristics, namely large engineering investment, difficult production control, and high technology content.

A phosgenation process for preparing TDI is still a mainstream process in industrial production. A synthetic reaction of TDI generally consists of five steps: (1) carbon monoxide reacts with hydrogen to generate phosgene; (2) toluene reacts with nitric acid to generate dinitrotoluene (DNT); (3) DNT reacts with hydrogen to generate toluene diamine (TDA); (4) the treated dried TDA reacts with phosgene to generate TDI; and (5) TDI is subjected to purification.

However, at present, a commonly used TDI production device is high in energy consumption, complex in structure, and low in energy utilization rate. In order to solve the above problems, the present disclosure provides a device for producing isocyanate by a light solvent method and a production method thereof.

### SUMMARY

In order to solve the above problems, the present disclosure provides a device for producing isocyanate by a light solvent method and a production method thereof, thereby achieving an objective of optimizing a process flow and improving energy efficiency.

In order to achieve the above objective, the present disclosure provides the following solution.

A device for producing isocyanate by a light solvent method, including a pre-fractionation column configured to introduce an isocyanate photochemical reaction material; and a main column configured to receive phosgene, a solvent, and isocyanate produced in the pre-fractionation column; where a top of the pre-fractionation column is in communication with a top of the main column, and a kettle of the pre-fractionation column is in communication with a kettle of the main column.

Preferably, the device further including a condenser arranged adjacent to the top of the main column, where the condenser is in circulation communication with the top of the main column.

Preferably, the device further includes a reboiler arranged at the kettle of the main column, and the reboiler is in circulation communication with the main column.

Preferably, the main column is provided with a side line for extraction.

Preferably, the solvent includes one or more of benzene, toluene, xylene, chlorobenzene, and dichlorobenzene.

Preferably, the top of the pre-fractionation column and the top of the main column, as well as the kettle of the pre-fractionation column and the kettle of the main column, are all in communication through pipelines.

Preferably, there are two pipelines between the top of the pre-fractionation column and the top of the main column, and two pipelines between the kettle of the pre-fractionation column and the kettle of the main column.

A method for producing isocyanate provided, including the following steps:
continuously introducing an isocyanate photochemical reaction material into a pre-fractionation column;
extracting phosgene and a gas-phase solvent from a top of the pre-fractionation column and then directly entering a top of a main column for condensation and collection; and extracting a liquid-phase solvent and isocyanate from a kettle of the pre-fractionation column and then directly entering a kettle of the main column for separation;
extracting a liquid-phase solvent with a purity of not less than 99.5% through a side line arranged on the main column, and extracting an isocyanate solution with a content of not less than 99.6% from the kettle of the main column; and
conveying the isocyanate solution to a process flow in the next stage for further purification.

Preferably, an isocyanate photochemical reaction material includes reaction solution containing isocyanate, phosgene, a solvent, and a reaction by-product, the isocyanate has a concentration of 15% to 16%, the phosgene has a concentration of 41% to 42%, and the solvent has a concentration of 42% to 45%.

Preferably, operating pressures of the pre-fractionation column and the main column are both 3 bara.

Compared with the prior art, the present disclosure has the following technical effects.

Compared with the prior art, the present disclosure combines a phosgene removal column and a first solvent removal column into one column to form a coupling system, that is, phosgene and a solvent are extracted from the top of the pre-fractionation column and directly introduced into the top of the main column; and a solvent and isocyanate are extracted from the kettle of the pre-fractionation column and then directly introduced into the kettle of the main column. The main column removes phosgene and the solvent from the kettle liquid by adjusting an appropriate kettle temperature, and then condensate at the top of the column is used as an absorbent in a phosgene recovery system by adjusting an appropriate top temperature. A liquid-phase solvent is extracted from a side line of the main column, and a crude isocyanate product separated from the kettle of the main column enters the next process. Compared with a conventional process, an entire coupling system combines two columns into one column, thereby reducing a reboiler and a condenser, avoiding a back-mixing effect of intermediate components, reducing a mixing problem caused by a difference in material composition between the feed and the stream on the feed tray, and saving 20 to 30% of energy consumption. The device is novel in design, simple in structure, and scientific in principle. By optimizing the process flow, a production cost is reduced, and energy utilization efficiency is also significantly improved, thereby meeting dual requirements of modern chemical production for environmental protection and economy.

### BRIEF DESCRIPTION OF THE DRAWINGS

In order to more clearly explain the technical solution in the present disclosure or the prior art, the drawings needed to be used in the embodiments will be briefly introduced hereinafter. Obviously, the drawings in the following description are only some embodiments of the present disclosure. For those skilled in the art, other drawings may be obtained according to these drawings without paying creative labor.

FIGURE is a schematic diagram of a structure according to the present disclosure.

1. Pre-fractionation column; 2. Main column; 3. Condenser; 4. Reboiler; 5. Gas-phase return pipe; 6. Liquid-phase return pipe; 7. Side line.

### DETAILED DESCRIPTION OF THE EMBODIMENTS

The technical solution in the embodiment of the present disclosure will be clearly and completely described with reference to the drawings in the embodiment of the present disclosure hereinafter. Obviously, the described embodiments are only some of the embodiments of the present disclosure, rather than all of the embodiments. Based on the embodiments in the present disclosure, all other embodiments obtained by those skilled in the art without paying creative labor belong to the scope of protection of the present disclosure.

In order to solve the above problems, the present disclosure provides a device for producing isocyanate by a light solvent method and a production method thereof, thereby achieving an objective of optimizing a process flow and improving energy efficiency.

To make the above objective, features and advantages of the present disclosure more obvious and understandable, the present disclosure will be further described in detail with reference to the drawings and the specific embodiments.

With reference to FIGURE, a device for producing isocyanate by a light solvent method includes a pre-fractionation column configured to introduce an isocyanate photochemical reaction material; and a main column configured to receive phosgene, a solvent, and isocyanate produced in the pre-fractionation column. A top of the pre-fractionation column is in communication with a top of the main column, and a kettle of the pre-fractionation column is in communication with a kettle of the main column. The present disclosure combines a phosgene removal column and a first solvent removal column into one column to form a coupling system, that is, phosgene and a solvent are extracted from the top of the pre-fractionation column and directly introduced into the top of the main column; and a solvent and isocyanate are extracted from the kettle of the pre-fractionation column and then directly introduced into the kettle of the main column. The main column removes phosgene and the solvent from the kettle liquid by adjusting an appropriate kettle temperature, and then condensate at the top of the column is used as an absorbent in a phosgene recovery system by adjusting an appropriate top temperature. A liquid-phase solvent is extracted from a side line of the main column, and a crude isocyanate product separated from the kettle of the main column enters the next process. Compared with a conventional process, an entire coupling system combines two columns into one column, thereby reducing a reboiler and a condenser, avoiding a back-mixing effect of intermediate components, reducing a mixing problem caused by a difference in material composition between the feed and the stream on the feed tray, and saving 20 to 30% of energy consumption. The device is novel in design, simple in structure, and scientific in principle. By optimizing the process flow, a production cost is reduced, and energy utilization efficiency is also significantly improved, thereby meeting dual requirements of modern chemical production for environmental protection and economy.

With reference to FIGURE, the device further includes a condenser arranged adjacent to the top of the main column, and the condenser is in circulation communication with the top of the main column.

With reference to FIGURE, the device further includes a reboiler arranged at the kettle of the main column, and the reboiler is in circulation communication with the main column.

With reference to FIGURE, the main column is provided with a side line for extraction.

Further, the solvent includes one or more of benzene, toluene, xylene, chlorobenzene, and dichlorobenzene.

With reference to FIGURE, the top of the pre-fractionation column and the top of the main column, as well as the kettle of the pre-fractionation column and the kettle of the main column, are all in communication through pipelines.

With reference to FIGURE, there are two pipelines between the top of the pre-fractionation column and the top of the main column, and two pipelines between the kettle of the pre-fractionation column and the kettle of the main column.

A method for producing isocyanate is described below with two examples about toluene diisocyanate.

### Example 1

Toluene diisocyanate (TDI for short) reaction solution containing TDI, phosgene, a solvent, and a reaction by-product is continuously introduced into a pre-fractionation column, in which the TDI has a concentration of 15.7%, the phosgene has a concentration of 41.3%, the solvent has a concentration of 42.96%, and the operating pressure of the column is 3 bara. Phosgene and a solvent are extracted from a top of the pre-fractionation column, and then directly enter a top of a main column. A solvent and TDI are extracted from a kettle of the pre-fractionation column, and then directly enter a kettle of the main column. The operating pressure of the column is also 3 bara. The gas phase at the top of the main column enters a condenser of the main column, and a condensation temperature is 40 °C. The mixed gas such as the solvent and phosgene at the top of the column is condensed at the same time to minimize or prevent phosgene from entering a vacuum system. Condensate at the top of the column is used as an absorbent in a phosgene recovery system. A liquid-phase solvent with a purity of 99.5% (mass fraction) is extracted from the side line of the main column. The kettle of the main column has a temperature of 170 °C. The TDI in the TDI reaction solution obtained from the kettle has a content of 96.64%. Then, the TDI product directly enters a process flow in the next stage for further purification. The main column is connected to a rectification section of the pre-fractionation column through a gas-phase reflux pipeline, and is connected to a stripping section of the pre-fractionation column through a liquid-phase reflux pipeline.

### Example 2

This example is the same as Example 1 except for the following features.

Toluene diisocyanate (TDI for short) reaction solution containing TDI, phosgene, a solvent, and a reaction by-product is continuously introduced into a pre-fractionation column, in which the TDI has a concentration of 16%, the phosgene has a concentration of 42%, the solvent has a concentration of 44%, and the operating pressure of the column is 3.2 bara. Phosgene and a solvent are extracted from a top of the pre-fractionation column, and then directly enter a top of a main column. A solvent and TDI are extracted from a kettle of the pre-fractionation column, and then directly enter a kettle of the main column. The operating pressure of the column is also 3.2 bara. The gas phase at the top of the main column enters a condenser of the main column, and a condensation temperature is 42 °C. The mixed gas such as the solvent and phosgene at the top of the column is condensed at the same time to minimize or prevent phosgene from entering a vacuum system. Condensate at the top of the column is used as an absorbent in a phosgene recovery system. A liquid-phase solvent with a purity of 99.8% (mass fraction) is extracted from the side line of the dividing wall column. The kettle of the main column has a temperature of 175 °C. The TDI in the TDI reaction solution obtained from the kettle has a content of 97.74%. Then, the TDI product directly enters a process flow in the next stage for further purification. The main column is connected to a rectification section of the pre-fractionation column through a gas-phase reflux pipeline, and is connected to a stripping section of the pre-fractionation column through a liquid-phase reflux pipeline.

Adaptive changes made according to actual needs are within the scope of protection of the present disclosure.

It should be noted that it is obvious to those skilled in the art that the present disclosure is not limited to details of the foregoing exemplary embodiments, and can be achieved in other specific forms without departing from the spirit or basic characteristics of the present disclosure. Therefore, the embodiments should be considered in all aspects as illustrative and not restrictive. The scope of the present disclosure is defined by the appended claims rather than the above description, and therefore, all changes that fall within the meaning and scope of equivalents of the claims is intended to be included in the present disclosure. Any reference numbers in the claims shall not be construed as limiting the claims concerned.

1. A device for producing isocyanate by a light solvent method, comprising
a pre-fractionation column configured to introduce an isocyanate photochemical reaction material;
and a main column configured to receive phosgene, a solvent, and isocyanate produced in the pre-fractionation column;
wherein a top of the pre-fractionation column is in communication with a top of the main column, and a kettle of the pre-fractionation column is in communication with a kettle of the main column.

2. The device for producing isocyanate by the light solvent method according to claim 1, further comprising a condenser arranged adjacent to the top of the main column, wherein the condenser is in circulation communication with the top of the main column.

3. The device for producing isocyanate by the light solvent method according to claim 2, further comprising a reboiler arranged at the kettle of the main column, wherein the reboiler is in circulation communication with the main column.

4. The device for producing isocyanate by the light solvent method according to claim 1, wherein the main column is provided with a side line for extraction.

5. The device for producing isocyanate by the light solvent method according to claim 1, wherein the solvent comprises one or more of benzene, toluene, xylene, chlorobenzene, and dichlorobenzene.

6. The device for producing isocyanate by the light solvent method according to claim 1, wherein the top of the pre-fractionation column and the top of the main column, as well as the kettle of the pre-fractionation column and the kettle of the main column, are all in communication through pipelines.

7. The device for producing isocyanate by the light solvent method according to claim 6, wherein there are two pipelines between the top of the pre-fractionation column and the top of the main column, and two pipelines between the kettle of the pre-fractionation column and the kettle of the main column.

8. A method for producing isocyanate, wherein the method is implemented by the device for producing isocyanate by the light solvent method according to claim 1, comprising the following steps:
continuously introducing an isocyanate photochemical reaction material into a pre-fractionation column,
extracting phosgene and a gas-phase solvent from a top of the pre-fractionation column and then directly entering a top of a main column for condensation and collection; and extracting a liquid-phase solvent and isocyanate from a kettle of the pre-fractionation column and then directly entering a kettle of the main column for separation;
extracting a liquid-phase solvent with a purity of not less than 99.5% through a side line arranged on the main column, and extracting an isocyanate solution with a content of not less than 99.6% from the kettle of the main column; and
conveying the isocyanate solution to a process flow in the next stage for further purification.

9. A method for producing isocyanate, wherein the method is implemented by the device for producing isocyanate by the light solvent method according to claim 2, comprising the following steps:
continuously introducing an isocyanate photochemical reaction material into a pre-fractionation column,
extracting phosgene and a gas-phase solvent from a top of the pre-fractionation column and then directly entering a top of a main column for condensation and collection; and extracting a liquid-phase solvent and isocyanate from a kettle of the pre-fractionation column and then directly entering a kettle of the main column for separation;
extracting a liquid-phase solvent with a purity of not less than 99.5% through a side line arranged on the main column, and extracting an isocyanate solution with a content of not less than 99.6% from the kettle of the main column; and
conveying the isocyanate solution to a process flow in the next stage for further purification.

10. A method for producing isocyanate, wherein the method is implemented by the device for producing isocyanate by the light solvent method according to claim 3, comprising the following steps:
continuously introducing an isocyanate photochemical reaction material into a pre-fractionation column,
extracting phosgene and a gas-phase solvent from a top of the pre-fractionation column and then directly entering a top of a main column for condensation and collection; and extracting a liquid-phase solvent and isocyanate from a kettle of the pre-fractionation column and then directly entering a kettle of the main column for separation;
extracting a liquid-phase solvent with a purity of not less than 99.5% through a side line arranged on the main column, and extracting an isocyanate solution with a content of not less than 99.6% from the kettle of the main column; and
conveying the isocyanate solution to a process flow in the next stage for further purification.

11. A method for producing isocyanate, wherein the method is implemented by the device for producing isocyanate by the light solvent method according to claim 4, comprising the following steps:
continuously introducing an isocyanate photochemical reaction material into a pre-fractionation column,
extracting phosgene and a gas-phase solvent from a top of the pre-fractionation column and then directly entering a top of a main column for condensation and collection; and extracting a liquid-phase solvent and isocyanate from a kettle of the pre-fractionation column and then directly entering a kettle of the main column for separation;
extracting a liquid-phase solvent with a purity of not less than 99.5% through a side line arranged on the main column, and extracting an isocyanate solution with a content of not less than 99.6% from the kettle of the main column; and
conveying the isocyanate solution to a process flow in the next stage for further purification.

12. A method for producing isocyanate, wherein the method is implemented by the device for producing isocyanate by the light solvent method according to claim 5, comprising the following steps:
continuously introducing an isocyanate photochemical reaction material into a pre-fractionation column,
extracting phosgene and a gas-phase solvent from a top of the pre-fractionation column and then directly entering a top of a main column for condensation and collection; and extracting a liquid-phase solvent and isocyanate from a kettle of the pre-fractionation column and then directly entering a kettle of the main column for separation;
extracting a liquid-phase solvent with a purity of not less than 99.5% through a side line arranged on the main column, and extracting an isocyanate solution with a content of not less than 99.6% from the kettle of the main column; and
conveying the isocyanate solution to a process flow in the next stage for further purification.

13. A method for producing isocyanate, wherein the method is implemented by the device for producing isocyanate by the light solvent method according to claim 6, comprising the following steps:
continuously introducing an isocyanate photochemical reaction material into a pre-fractionation column,
extracting phosgene and a gas-phase solvent from a top of the pre-fractionation column and then directly entering a top of a main column for condensation and collection; and extracting a liquid-phase solvent and isocyanate from a kettle of the pre-fractionation column and then directly entering a kettle of the main column for separation;
extracting a liquid-phase solvent with a purity of not less than 99.5% through a side line arranged on the main column, and extracting an isocyanate solution with a content of not less than 99.6% from the kettle of the main column; and
conveying the isocyanate solution to a process flow in the next stage for further purification.

14. A method for producing isocyanate, wherein the method is implemented by the device for producing isocyanate by the light solvent method according to claim 7, comprising the following steps:
continuously introducing an isocyanate photochemical reaction material into a pre-fractionation column,
extracting phosgene and a gas-phase solvent from a top of the pre-fractionation column and then directly entering a top of a main column for condensation and collection; and extracting a liquid-phase solvent and isocyanate from a kettle of the pre-fractionation column and then directly entering a kettle of the main column for separation;
extracting a liquid-phase solvent with a purity of not less than 99.5% through a side line arranged on the main column, and extracting an isocyanate solution with a content of not less than 99.6% from the kettle of the main column; and
conveying the isocyanate solution to a process flow in the next stage for further purification.

15. The method for producing isocyanate according to claim 8, wherein an isocyanate photochemical reaction material comprises reaction solution containing isocyanate, phosgene, a solvent, and a reaction by-product, the isocyanate has a concentration of 15% to 16%, the phosgene has a concentration of 41% to 42%, and the solvent has a concentration of 42% to 45%.

16. The method for producing isocyanate according to claim 8, wherein operating pressures of the pre-fractionation column and the main column are both 3 bara.
